(19) United States
(12) Patent Application Publication
(10) Pub. No.: US 2003/0157664 A1
(43) Pub. Date: Aug. 21, 2003

(54) Novel mutants of the formate dehydrogenase from Candida boidinii

(75) Inventors: Heike Slusarczyk, Uebach-Palenberg (DE); Stephan Felber, Juelich (DE); Maria-Regina Kula, Muenchen (DE); Martina Pohl, Aachen (DE)

Correspondence Address: OBLON, SPIVAK, MCCLELLAND, MAIER & NEUSTADT, P.C., FOURTH FLOOR, 1755 JEFFERSON DAVIS HIGHWAY, ARLINGTON, VA, 22202, US

(73) Assignee: Degussa AG, Duesseldorf (DE)

(21) Appl. No.: 10/252,086

(22) Filed: Sep. 23, 2002

(30) Foreign Application Priority Data
Sep. 21, 2001 (DE) 101 46 589.0

(51) Int. Cl. C12P 13/04; C12P 7/02; C12N 9/02; C12N 1/18; C07H 21/04; C12P 21/02; C12N 15/74

(52) U.S. Cl. 435/106; 435/155; 435/69.1; 435/189; 435/254.22; 435/320.1; 536/23.2

(57) ABSTRACT

Novel improved enzymes prepared by recombination, especially to rec-FDHs. Directed evolution has made it possible to generate catalytically more active and more stable muteins which can preferably be used in an industrial process for the preparation of e.g. amino acids. The invention further relates to the nucleic acids coding for these enzymes, to vehicles containing these nucleic acids and to advantageous primers for the preparation of the nucleic acids by means of PCR. The invention additionally relates to a process for the preparation of further improved rec-FDHs, and a method of screening more stable and/or more active dehydrogenases is claimed.

### BACKGROUND OF THE INVENTION

1. Field of the Invention

The present invention relates to novel mutants of a rec-FDH from Candida boidinii (ATCC 32195). The invention also describes the nucleic acids coding for these mutants and vehicles containing these nucleic acids. The invention also relates to a process for the preparation of further improved FDHs and to a process for the advantageous screening of more stable and more active dehydrogenases.

### BACKGROUND OF THE INVENTION

Biocatalysts, inter alia, are successfully used for the preparation of L-amino acids, a starting point being the conversion of prochiral α-keto acids by reductive amination. The amino acid dehydrogenases used in this reaction require stoichiometric amounts of NADH or NADPH as coenzyme to convert the α-keto acids. These coenzymes are very expensive and therefore render the above-mentioned process of little economic value for the industrial scale.

A possible way of avoiding high costs due to the coenzyme consists in regenerating the coenzyme in situ. At the present time, the NAD-dependent formate dehydrogenase from the yeast Candida boidinii, inter alia, is used on the industrial scale to regenerate the coenzyme in the enzyme reactor.

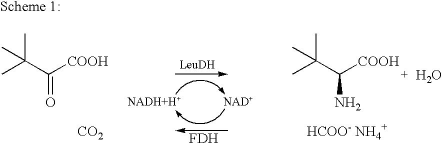

Scheme 1 shows the in situ regeneration of NADH with NAD-dependent formate dehydrogenase in the reductive amination of trimethyl pyruvate to L-tert-leucine (Bommarius et al., Tetrahedron Asymmetry 1995, 6, 2851-2888).

One disadvantage associated with the use of the FDH from Candida boidinii in the production process is the need to make up the FDH during the process because it loses activity due to a lack of stability.

This inactivation can be influenced by a variety of factors:

pH

temperature

mechanical loading

ionic strength and type of ions in the substrate solution

traces of heavy metals

oxidation of sulfhydryl groups by atmospheric oxygen

crosslinking due to thiol-disulfide exchange

Tishkov et al. showed that directed mutation of the recombinant FDH from Pseudomonas sp. 101 increased its stability towards mercury salts, although the mutagenesis reduced the thermal stability (Biochem. Biophys. Res. Commun. 1993, 192, 976-981).

Sakai et al. elucidated the gene sequence of the FDH from the methylotrophic yeast Candida boidinii (J. Bacteriol. 1997, 179, 4480-4485), the derived protein sequence being 100% identical to the amino acid sequence of the recombinant FDH from Candida boidinii.

As well as other mutants of the formate dehydrogenase from Candida boidinii, DE 19753350 describes the protein provided with serine as amino acid in position 23 (C23S) and the protein additionally provided with alanine in position 262 (C23S/C262A). In respect of the sensitivity to aggregation and oxidation, these proteins exhibit a higher stability than the native enzyme or the enzyme prepared by recombination (rec-), but do not possess an increased catalytic activity or thermal stability.

All NAD-dependent formate dehydrogenases described hitherto (EC 1.2.1.2) are characterized by relatively low specific activities of between 5 and 7 U/mg of protein at 30° C. (Popov, V. O., Lamzin, V. S. (1994) NAD⁺-dependent formate dehydrogenase. Biochem. J. 130, 625-643). By comparison, the leucine dehydrogenase (LeuDH) used for the preparation of L-tert-leucine (Scheme 1) has a specific activity of 200 U/mg. Thus, to assure a stoichiometric regeneration of NADH, it is necessary to provide and use many times the amount of FDH protein compared with LeuDH.

### SUMMARY OF THE INVENTION

In view of the state of the art reported and discussed above, it was therefore an object of the present invention further to increase both the catalytic activity and the stability of the more oxidation- and aggregation-insensitive rec-FDH from Candida boidinii with the C23S mutation or the C23S/C262A double mutation in order to have to prepare and use smaller amounts of enzyme for an industrial process and avoid an expensive making-up of the FDH during the process, thereby helping to save production costs.

Thus, it is an object of the present invention to provide the mutants described above.

It is another object to provide nucleic acids which encode the mutants, as well as vectors containing these nucleic acids.

It is another object of the invention to provide novel primers.

It is another object of the present invention to provide a process for the preparation of further improved muteins based on the mutants described herein, to the enzymes obtained by such a process, to nucleic acids coding for such enzymes, and to their use.

It is another object of the invention to provide a whole cell catalyst (i.e., a cell, especially a transformed cell).

It is also an object of the invention to provide a screening method for more active and more stable dehydrogenases.

The provision of mutants which are more stable and/or more catalytically active towards the wild-type rec-FDH and the native wild-type enzyme from Candida boidinii, said mutants containing the amino acid exchange C23S (DE 19753350; SEQ ID NO: 1) or C23S/C262A (DE 19753350; SEQ ID NO: 3) as well as one or more of the following amino acid exchanges: E18D, K35R, D149E, E151D, R178S, R178G, K206R, F285Y, F285S, T315N and K356E, affords improved biocatalysts which can advantageously be used e.g. in an industrial process as mentioned at the outset, or in a process for the preparation of chiral compounds, especially amino acids (natural and unnatural) in optically enriched (enantiomer-enriched) form. Optionally the mutants can additionally contain the C262A mutation.

A more complete appreciation of the invention and many of the attendant advantages thereof will be readily obtained as the same becomes better understood by reference to the following Figures in conjunction with the detailed description below.

### BRIEF DESCRIPTION OF THE FIGURES

FIG. 1: mean inactiviating temperatures of mutants described in the Examples herein.

FIG. 2: restriction map of pBTac-FDH-C23S (5676 bp).

FIG. 3: restriction map for pUC-FDH (FIG. 3).

### DETAILED DESCRIPTION OF THE INVENTION

One skilled in the art will readily appreciate how to introduce these advantageous amino acid exchanges according to the invention into other FDHs which do not originate from Candida boidinii but which have a correspondingly homologous sequence. The invention therefore further relates to amino acid sequences with FDH activity which are more stable and/or more active towards the wild-type rec-FDH and the native wild-type enzyme from Candida boidinii and which contain one or more of the following amino acid exchanges: 18D, 35R, 149E, 151D, 178S, 178G, 206R, 285Y, 285S, 315N and 356E, the exchanges taking place in the corresponding equivalent positions in the sequence.

The position numbers of amino acids are assigned by continuous numbering of the amino acids beginning with the start codon of the sequence. Therefore, if there are deletions or insertions, amino acids which influence the enzyme function in the same way can have quite different position numbers in enzymes of the same type. Similarly, an equivalent position in enzymes of the same type contributes to the change in activity and stability, as in the C23S starting mutant. Provided there is a high sequence homology of e.g. >60% between the enzymes of the same type, the corresponding equivalent positions between the mutant from Candida boidinii and the amino acid sequence to be mutated can be identified by so-called alignment, e.g. with the BLAST® program (J. Mol. Biol. 1990, 215, 403-410). In this method, conserved regions in the sequences to be compared are placed directly underneath one another. A corresponding equivalent position is obtained from the positions on the reference strand which correspond to the positions indicated above, taking into account the fact that the exchange in said position makes a similar contribution to the change in activity and stability as it does in the C23S mutant. Another possible way of identifying equivalent positions is to compare X-ray structural studies (Cur. Opin. Struc. Biol. 1995, 5, 377-382), such positions being identifiable by superimposing 3D structures of enzymes. Empirical and semiempirical structural analysis programs can assist in this context. The exchanges in the amino acid sequences with FDH activity can be effected by mutagenesis methods indicated below, which are familiar to those skilled in the art, and by the use of recombinant techniques (see below for bibliography).

The invention further relates to nucleic acids having a sequence coding for a mutant of FDH from C. boidinii, according to the invention, and to the above-mentioned amino acid sequences with FDH activity. These also include nucleic acids according to the invention which, in addition to the actual coding sequence, contain e.g. the sequences important for restriction enzymes, Tag sequences (His-, Mal-) or transcription terminators (rrnB).

The details of the nucleic acids advantageously provide access to substances which make it possible to assure an adequate amount of the enzymes necessary for an enzyme-based industrial process, as mentioned at the outset, for the production of e.g. amino acids. Via known recombinant techniques (see below) it is possible, with the nucleic acids according to the invention, to recover high yields of the enzymes from fast-growing host organisms. Moreover, the gene sequences according to the invention can be used to produce mutants which may exhibit further improvements. Said recombinant techniques, with which those skilled in the art are sufficiently familiar (see below), provide access to organisms which are capable of providing the enzyme in question in an amount adequate for an industrial process. The rec-enzymes according to the invention are prepared by genetic engineering methods known to those skilled in the art (Sambrook et al. 1989, Molecular Cloning: A Laboratory Manual, 2nd edition, Cold Spring Harbor Laboratory Press; Balbas P. & Bolivar F. 1990, Design and construction of expression plasmid vectors in E. coli, Methods Enzymology 185, 14-37; Vectors: A Survey of Molecular Cloning Vectors and Their Uses. R. L. Rodriguez & D. T. Denhardt, eds: 205-225). As regards the general procedure (PCR, cloning, expression etc.), reference may also be made to the following literature and the material cited therein: Sambrook J., Fritsch E. F., Maniatis T. (1989). Molecular Cloning. Cold Spring Harbor Laboratory Press; Vectors: A Survey of Molecular Cloning Vectors and Their Uses. R. L. Rodriguez & D. T. Denhardt, II.

The invention further relates to plasmids or vectors containing one or more of the nucleic acids according to the invention.

In principle, suitable plasmids or vectors are any of the variants available for this purpose to those skilled in the art. Such plasmids and vectors can be found in Studier et al., Methods Enzymol. 1990, 185, 61-69, or in the brochures issued by Roche Biochemicals, Invitrogen, Novagen, Promega, New England Biolabs, Clontech or Gibco BRL. Particularly preferred plasmids and vectors can be found in DNA cloning: a practical approach, Volume I-III, edited by D. M. Glover, IRL Press Ltd., Oxford, Washington D.C., 1985, 1987; Denhardt, D. T. and Colasanti, J.: A survey of vectors for regulating expression of cloned DNA in E. coli. In: Rodriguez, R. L. and Denhardt, D. T. (eds). Vectors, Butterworth, Stoneham, Mass., 1987, pp. 179-204; Gene expression technology. In: Goeddel, D. V. (eds), Methods in Enzymology, Volume 185, Academic Press, Inc., San Diego, 1990; Sambrook, J., Fritsch, E. F. and Maniatis, T. 1989. Molecular cloning: a laboratory manual, 2nd ed. Cold Spring Harbor Laboratory Press, Cold Spring Harbor, N.Y.

Plasmids with which the gene construct containing the nucleic acid according to the invention can very particularly preferably be cloned into the host organism are: pKK-177-3H (Roche Biochemicals), pBTac (Roche Biochemicals), pKK-233-3 (Amersham Pharmacia Biotech), pLex (Invitrogen) or the vectors of the pET series (Novagen).

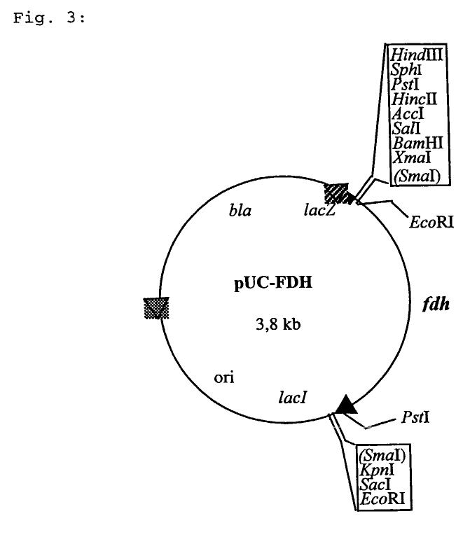

Plasmids pBTac-FDH (FIG. 2) and pUC-FDH (FIG. 3) are exceedingly preferred.

The invention likewise relates to microorganisms containing the nucleic acids according to the invention.

The microorganism into which the nucleic acids are cloned is used for increasing and recovering a sufficient amount of the recombinant enzyme. The relevant processes are well known to those skilled in the art (Sambrook et al. 1989, Molecular cloning: A Laboratory Manual, 2nd Edition, Cold Spring Harbor Laboratory Press; Balbas P. & Bolivar F. 1990, Design and construction of expression plasmid vectors in E. coli, Methods Enzymology 185, 14-37; and above-cited bibliography relating to recombinant techniques). In principle, the microorganisms used can be any of the organisms used for this purpose by those skilled in the art, e.g. prokaryotes or eukaryotes such as Pseudomonas, Streptomyces, Arthrobacter, Bacillus, Staphylococcus, Escherichia, Candida, Hansenula, Pichia and baculoviruses. It is preferable to use E. coli strains for this purpose, the following being very particularly preferred: E. coli NM 522, XL1 Blue, JM101, JM109, JM105, RR1, DH5α, TOP 10⁻ or HB101. Plasmids with which the gene construct containing the nucleic acid according to the invention is preferably cloned into the host organism are indicated above.

A further feature of the invention concerns primers for the preparation of the gene sequences according to the invention by means of all kinds of PCR. They include the sense and antisense primers coding for the corresponding amino acid sequences.

In principle, suitable primers can be obtained by methods known to those skilled in the art. The primers according to the invention are identified by comparison with known DNA sequences or by translation of the contemplated amino acid sequences into the codon of the organism in question (e.g. for Streptomyces: Wright et al., Gene 1992, 113, 55-65). Common characteristics in the amino acid sequence of proteins of so-called superfamilies are also useful for this purpose (Firestine et al., Chemistry & Biology 1996, 3, 779-783). Further information on this subject can be found in Oligonucleotide synthesis: a practical approach, edited by M. J. Gait, IRL Press Ltd, Oxford, Washington D.C., 1984; PCR Protocols: A guide to methods and applications, edited by M. A. Innis, D. H. Gelfound, J. J. Sninsky and T. J. White. Academic Press, Inc., San Diego, 1990. Primers which can simultaneously introduce the sequences important for restriction enzymes into the nucleic acid sequence to be synthesized are also preferred.

The following primers are very particularly preferably to be used for producing the mutants:

TABLE 1

| Primer name | Seq. | Seq. no. |
|---|---|---|
| PBTACF1 | 5′ TGC CTG GCA GTT CCC TAC TC 3′ | 25 |
| PBTACF2 | 5′ CGT TTC ACT TCT GAG TTC GG 3′ | 26 |
| PBTACR1 | 5′ GGT ATG GCT GTG CAG GTC GT 3′ | 27 |
| PBTACR2 | 5′ CGA CAT CAT AAC GGT TCT GG 3′ | 28 |
| PBTACR3 | 5′ TCA TCG GCT CGT ATA ATG TG 3′ | 29 |
| F285S-F1 | 5′- GGT GAT GTT TGG TCC CCA CAA CCA GCT CCA AAG -3′ | 30 |
| F285S-R1 | 5′- GGA GCT GGT TGT GGG GAC CAA ACA TCA CCG TA -3′ | 31 |

The invention also relates to a process for the preparation of further improved rec-FDHs from nucleic acids coding for one of the rec-FDH mutants according to the invention, wherein

a) the nucleic acids are subjected to a mutagenesis,

b) the nucleic acids obtainable from a) are cloned into a suitable vector and the latter is transferred into a suitable expression system, and

c) the improved proteins formed are detected and isolated.

This process can be carried out once or any desired number of times in succession.

Those skilled in the art are sufficiently familiar with the procedure for improving the enzymes according to the invention by mutagenesis methods. Suitable mutagenesis methods are any of the methods available for this purpose to those skilled in the art, especially saturation mutagenesis (A. R. Oliphant, A. L. Nussbaum, K. Struhl (1986) Cloning of random sequence oligonucleotides, Gene 44, 177-183), random mutagenesis (R. C. Caldwell, G. F. Joyce (1992) Randomization of genes by PCR mutagenesis, PCR Methods Appl. 2, 28-33), recombination methods such as shuffling (W. P. Stemmer (1994) DNA shuffling by random fragmentation and reassembly: In vitro recombination for molecular evolution, Proc. Natl. Acad. Sci. USA 91, 10747-10751), L-shuffling (EP 1104457) or StEP (H. Zhao, L. Giver, Z. Shao, J. Affholter, F. Arnold (1998) Molecular evolution by staggered extension process (StEP) in vitro recombination, Nat. Biotechnol. 16, 258-261), and site-directed mutagenesis (S. N. Ho, H. D. Hunt, R. M. Horton, J. K. Pullen, L. R. Pease (1989) Site-directed mutagenesis by overlap extension using the polymerase chain reaction, Gene 77, 248-254). The novel nucleic acid sequences obtained are cloned into a host organism by the methods described above (see above for bibliography) and the expressed enzymes are detected by suitable screening methods (especially for FDH analysis, see below).

Another feature according to the invention concerns the improved rec-FDHs obtainable by a process as described above, and the nucleic acids coding for these rec-FDHs.

The invention further relates to the use of the rec-FDHs according to the invention, optionally improved by mutation, for the preparation of chiral enantiomer-enriched organic compounds, e.g. alcohols or amino acids.

Furthermore, the further improved nucleic acids according to the invention, coding for the rec-FDHs in question, are preferentially suitable for the preparation of whole cell catalysts (DE 10037115.9 and bibliography cited therein).

The invention thus also provides a whole cell catalyst containing a cloned gene for a dehydrogenase and a cloned gene for a rec-FDH, preferably a rec-FDH from Candida boidinii and particularly preferably a rec-FDH according to the invention.

Those skilled in the art are familiar with the preparation of such an organism (PCT/EP00/08473; PCT/JUS00/08159; see below for bibliography).

The advantage of such an organism is the simultaneous expression of both enzyme systems even though only one rec-organism has to be used for the reaction. To adjust the expression of the enzymes in respect of their reaction rates, it is possible to accommodate the appropriately coding nucleic acid fragments on different plasmids with different copy numbers and/or to use promoters of different strengths for gene expressions of different strengths. By adjusting enzyme systems in this way, there is advantageously no accumulation of an intermediate possibly having an inhibitory action, and the reaction in question can proceed at an optimum overall rate, this being sufficiently familiar to those skilled in the art (PCT/EP00/08473; Gellissen et al., Appl. Microbiol. Biotechnol. 1996, 46, 46-54).

Another feature of the present invention, which is no less advantageous, concerns a method of identifying more active mutants of an NAD- or NADP-dependent dehydrogenase, comprising a quantitative screening method for determination of the activity, said method specifically consisting of the following steps:

a) equal aliquots of the cell digesting solution of the mutants to be compared are brought into contact with equal amounts of affinity chromatography material (solid phase),

b) the affinity chromatography material is separated from the non-adhering constituents,

c) the muteins adhering to the affinity chromatography material are eluted, and

d) the volume activity and protein concentration, and hence the specific activity, are determined.

The method according to the invention makes it possible to determine the specific activity based on the amount of enzyme (volume activity/protein concentration) in a particularly simple manner.

The determination is preferably performed directly on so-called microtitre plates. Specifically, the screening procedure consists initially in carrying out a qualitative detection process directly on an agar plate, where positive muteins are identified directly on the agar plate by means of an activity dye in the presence of formate, phenazine ethosulfate, nitrotetrazolium blue chloride and NAD (modified according to O. Gabriel, J. Mol. Biol. 1971, 22, 578-604; A. S. Hawrany et al., J. Mol. Biol. 1996, 264, 97-110). Active clones are picked from the agar plates and cultivated in 96-well microtitre plates. Subsequent expression of the FDH muteins is induced by adding the inducer isopropyl thiogalactoside (IPTG).

After an average of 16 hours of expression, the cytoplasmic FDH activities are released by treating the cell suspension with Triton-X100/EDTA in order to render the cells permeable. The FDH muteins are isolated and purified to the point of homogeneity in microtitre plates by means of one-step affinity chromatography (K. H. Kroner et al., J. Chem. Tech. Biotechnol. 1982, 32, 130-137; N. E. Labrou et al., Arch. Biochem. Biophys. 1995, 32, 169-178), the cell digesting solution being brought into contact with the affinity chromatography material in a batch process (Procion Red HE-3B, Sigma; bound to Streamline AC, Pharmacia) and non-adhering fractions in the supernatant being withdrawn. Specific elution of the FDH then followed with NAD.

Affinity chromatography materials which may be mentioned are any of the materials which can be used for this purpose by those skilled in the art and which are capable of selectively binding dehydrogenases. It is preferable to use sepharose, particularly red or blue sepharose (A. Walsdorf, D. Forciniti, M.-R. Kula (1990) Investigation of affinity partition chromatography using formate dehydrogenase as a model. J. Chromatography 523, 103-117; U. Reichert, E. Knieps, H. Slusarczyk, M.-R. Kula, J. Thömmes (2001) Isolation of a recombinant formate dehydrogenase by pseudo affinity expanded bed adsorption, J. Biochem. Biophys. Methods, in press; N. E. Labrou, Y. D. Clonis, (1995) The interaction of Candida boidinii formate dehydrogenase with a new family of chimeric biomimetic dye-ligands. Arch. Biochem. Biophys. 316(1), 169-178).

The FDH activity released (=initial activity Ao) is finally determined by means of the known photometric detection method (see above for bibliography) at 340 nm and 30° C. in a microtitre plate reader and related to the protein concentration determined according to Bradford et al. (Example 7.2).

In a parallel or subsequent operation, the stability can also be determined from the purified fractions. The stability was checked by incubating aliquots of the enzyme samples in the 96-well format in a PCR apparatus (Primus 96, MWG Biotech AG) for 15 min at a defined temperature (50° C. to 58° C.) which depended on the initial stability of the parent mutants of the respective generation. The residual activity (A15) was then determined at 30° C. in the standard assay (Examples 6 and 7.2). The quotient (Ti) of residual activity (A15) to initial activity (Ao) is a measure of the stability of the enzymes and is preferably used for stability analyses in the screening. A Ti value which is higher than that of the starting enzyme (e.g. FDH-C23S) means that the mutein studied has a higher stability.

The screening process for increasing the stability is illustrated in greater detail in the Examples and is sufficiently familiar to those skilled in the art (H. Zhao, F. Arnold (1999) Directed evolution converts subtilisin E into a functional equivalent of thermitase, Prot. Eng. 12(1), 47-53).

The screening systems described above can be used to screen more than 200,000 clones manually for FDH activity and/or stability in a very short time.

The random introduction of point mutations into the gene of the FDH-C23S or FDH-C23S/C262A mutant was effected using the error prone polymerase chain reaction technique known to those skilled in the art (Caldwell, R. C., Joyce, G. F. (1992) Randomization of Genes by PCR Mutagenesis. PCR Methods Appl. 2, 28-33).

For the successful error prone PCR of a gene segment and the sequencing of the mutants, five primers (PBTACF1, PBTACF2 and PBTACR1, PBTACR2, PBTACR3) were constructed.

Manganese chloride concentrations of 0.15 mM and 0.5 mM were used in the PCR preparation in order to adjust the error frequency. A 1500 bp fragment of each of the genes according to the invention was amplified by means of the PCR technique with the two outer primers PBTACF1 and PBTACR1 or PBTACR2. The base pair sequence of the FDH-C23S mutant, which, cloned in vector pBTac2, served as template in the first error prone PCR, is shown in Seq. 1 by way of example.

However, the cloned FDH genes of the mutants according to the invention advantageously also have an EcoRI restriction cleavage site at the 5′ end and a PstI cleavage site at the 3′ end, which have been added to the coding nucleotide sequence by means of PCR using the primers N-EcoRI and C-PstI in order to facilitate a subsequent directed cloning of the gene into vector pBTac2.

This was followed by a cloning of the whole DNA population of 1.1 kb EcoRI/PstI fragments into plasmid pBTac2 previously restricted with the restriction enzymes EcoRI and PstI. The resulting vectors were then transformed in E. coli to produce a mutant library. The FDH muteins from Candida boidinii can be overexpressed in E. coli JM101 or E. coli JM105 by means of expression plasmid pBTac-FDH (FIG. 2). The cloning and expression techniques are familiar to those skilled in the art and are described inter alia in Sambrook et al. (see above).

Successful mutations were found at the following sites:

TABLE 2
Mutations in the fdh-C23S gene or fdh-C23S/C262A gene which led
to an improvement in stability or activity.

| | Codon in FDH- | | |
|---|---|---|---|
| Mutation | C23S | Codon in mutant | Mutation in base pair |
| E18D | gaa | gac | 53 |
| K35R | aaa | aga | 104 |
| D149E | gat | gag | 447 |
| E151D | gag | gat | 453 |
| R178S | aga | agt | 534 |
| R178G | aga | gga | 532 |
| K206R | aaa | aga | 617 |
| F285Y | ttc | tac | 854 |
| F285S | ttc | tcc | 854 |
| T315N | act | aat | 944 |
| K356E | aaa | gaa | 1066 |

TABLE 3
Mutations in the mutants according to the invention which led to an
improvement in stability (FDH-C23S (SM) and FDH-C23S/C262A (DM) were the
templates (starting mutants) for the directed evolution)

| | Effect | | | | | |
|---|---|---|---|---|---|---|
| | Mean | Stability | | Codon in | | |
| | inactiva- | increase | | FDH-C23S | Codon | Mutation |
| | ting | relative to | | or FDH- | in | in base |
| Name | temp. [° C.] | parents [° C.] | Mutation | C23S/C262A | mutant | pair |
| FDH- | 47 | — | C23S | tct | tct | — |
| C23S/C262A | | | C262A | gct | gct | — |
| (DM) | | | | | | |
| FDH-C23S | 52 | — | C23S | tct | tct | — |
| (SM) | | | | | | |
| DM-3bE10 | 51 | 4 | C23S | tct | tct | — |
| | | | K206R | aaa | aga | 617 |
| | | | C262A | gct | gct | — |
| | | | T315N | act | aat | 944 |
| | | | K356E | aaa | gaa | 1066 |
| DM-2kA6 | 51 | 4 | E18D | gaa | gac | 53 |
| | | | C23S | tct | tct | — |
| | | | K35R | aaa | aga | 104 |
| | | | R178S | aga | agt | 534 |
| | | | C262A | gct | gct | — |
| DM-2hG12 | 58 | 7 | E18D | gaa | gac | 53 |
| | | | C23S | tct | tct | — |
| | | | K35R | aaa | aga | 104 |
| | | | E151D | gag | gat | 453 |
| | | | R178S | aga | agt | 534 |
| | | | C262A | gct | gct | — |
| | | | F285Y | ttc | tac | 854 |
| SM-1kA2 | 55 | 3 | C23S | tct | tct | — |
| | | | R178S | aga | agt | 534 |
| SM-1eA6 | 54 | 2 | C23S | tct | tct | — |
| | | | R178G | aga | gga | 532 |
| SM-2pC7 | 57 | 2 | C23S | tct | tct | — |
| | | | D149E | gat | gag | 447 |
| | | | R178S | aga | agt | 534 |
| SM-4cA10 | 62 | 4-11 | C23 | tct | tct | — |
| | | | E151D | gag | gat | 453 |
| | | | R178S | aga | agt | 534 |
| | | | K206R | aaa | aga | 617 |
| | | | T315N | act | aat | 944 |
| SM-4fD3 | 61 | 3-10 | C23S | tct | tct | — |
| | | | E151D | gag | gat | 453 |
| | | | R178S | aga | agt | 534 |
| SM-4sG4 | 59 | 1-8 | C23S | tct | tct | — |
| | | | E151D | gag | gat | 453 |
| | | | R178S | aga | agt | 534 |
| | | | K356E | aaa | gaa | 1066 |
| SM-4sG6 | 60 | 2-9 | C23S | tct | tct | — |
| | | | E151D | gag | gat | 453 |
| | | | R178S | aga | agt | 534 |
| | | | K206R | aaa | aga | 617 |
| | | | K356E | aaa | gaa | 1066 |

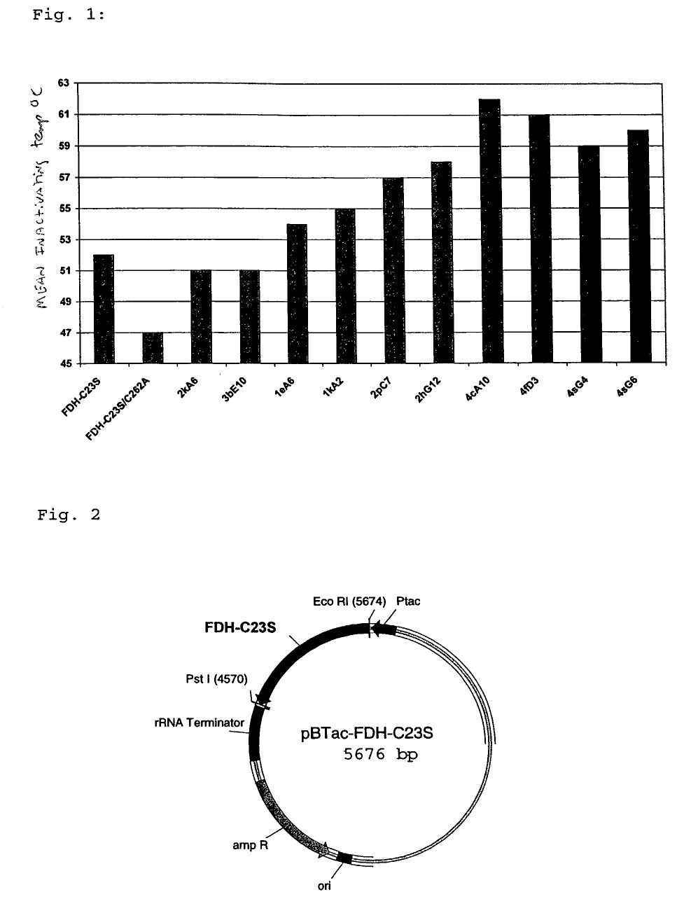

See FIG. 1.

TABLE 4
Mutations in the mutants according to the invention which effect an increase in activity

| | | | | Codon in | | Mutation |
|---|---|---|---|---|---|---|
| | Effect | Mean | | FDH-C23S or | Codon | in |
| | Catalytic | inactiva-ting | | FDH- | in | base |
| Name | activity | temp. [° C.] | Mutation | C23S/C262A | mutant | pair |
| FDH- | 1.7-fold | 52 | C23S | tct | tct | — |
| C23S/F285S | | | F285S | ttc | tcc | 854 |

The mutants exhibited the following improved properties:

1. The mean inactivating temperature of the most stable muteins is 9° C.-110° C. higher than that of the FDH-C23S starting mutant and 14° C.-15° C. higher than that of the FDH-C23S/C262A starting mutant.

2. The half-life at 54° C. of the most stable muteins is up to 200 times longer than that of the FDH-C23S starting mutant and up to 1000 times longer than that of the FDH-C23S/C262A starting mutant.

3. Through the directed evolution of FDH-C23S, the catalysis constant was increased from 3.7 s⁻¹ to 6.1 s⁻¹, i.e. by a factor of 1.7. Consequently the specific activity of the enzyme also increased from 5.5 U/mg to 9.1 U/mg. This shows that the more active mutant is twice as active as the FDH-C23S starting mutant and the recombinant wild-type enzyme.

4. The muteins can thus be prepared inexpensively in a high cell density fermentation of the recombinant E. coli strain JM101.

5. By introducing the F285S mutation into the SM-4fD3 mutant by means of site-specific mutagenesis, a mutant, SM-4fD3-F285S, was advantageously produced which exhibits both an increased activity and an increased stability.

By treating the already more oxidation- and aggregation-insensitive FDI™-C23S and FDH-C23S/C262A mutants by means of directed evolution using error prone PCR and site-specific mutagenesis, information is obtained about preferred positions for amino acid exchanges in the enzyme and about the type of amino acid which is preferably to be used. The enzymes mutated in this way possess higher activities and longer lives and thus give rise to lower enzyme consumption indices in the industrial process. Further improvements can be obtained by means of specific combinations of the individual mutations. With the novel mutants and their further developments, it is therefore possible considerably to improve e.g. the industrial process indicated at the outset.

For the application according to the invention, the enzymes in question can be used in the free form as homogeneously purified compounds. Furthermore, the enzyme can also be used as a constituent of an intact guest organism or in conjunction with the digested host organism cell mass purified to any desired degree. It is also possible to use the enzymes in an immobilized form (Bhavender P. Sharma, Lorraine F. Bailey and Ralph A. Messing, “Immobilisierte Biomaterialien—Techniken und Anwendungen”, Angew. Chem. 1982, 94, 836-852). The immobilization is advantageously effected by lyophilization (Dordick et al., J. Am. Chem. Soc. 1994, 116, 5009-5010; Okahata et al., Tetrahedron Lett. 1997, 38, 1971-1974; Adlercreutz et al., Biocatalysis 1992, 6, 291-305). Lyophilization in the presence of surface-active substances, such as Aerosol OT, polyvinylpyrrolidone, polyethylene glycol (PEG) or Brij 52 (diethylene glycol monocetyl ether), is very particularly preferred (Goto et al., Biotechnol. Techniques 1997, 11, 375-378). It is also conceivable to use the enzymes as CLECs (St Clair et al., Angew. Chem. Int. Ed. Engl. 2000 Jan, 39(2), 380-383).

Within the framework of the invention, optically enriched (enantiomer-enriched, enantiomerically enriched) compounds are understood as indicating the presence of one optical antipode in a mixture with the other in a proportion of >50 mol %.

A natural amino acid is an amino acid as described in Beyer-Walter, Lehrbuch der organischen Chemie, S. Hirzel Verlag Stuttgart, 22nd edition, 1991, p. 822 et seq. However, corresponding unnatural α-amino acids, such as those listed e.g. in DE 19903268.8, are also mentioned.

The organism Candida boidinii is deposited in the American Type Culture Collection under the number ATCC 32195 and is accessible by the public.

The term ‘nucleic acids’ encompasses both DNA and RNA.

The term ‘more active’ used in the present specification is understood according to the invention as meaning that the specific activity (based on the amount of FDH protein) is increased.

Within the framework of the invention, the expression ‘based on the stability of the enzymes’ refers primarily to the so-called thermal stability, which is a measure of the physical stability of a protein. This is specifically understood as meaning maintenance of the catalytic activity, i.e. maintenance of the active conformation of the enzyme at elevated temperatures (A. M. Klibanov, T. J. Ahern (1987) Thermal stability of proteins. In: Protein Engineering (D. L. Oxender, ed.), Alan R. Liss, Inc., 213-218). An increased thermal stability is understood according to the invention as meaning in particular that the half-life is increased at a given temperature (H. Zhao, F. H. Arnold (1999) Directed evolution converts subtilisin E into a functional equivalent of thermitase, Prot. Eng. 12(1), 47-53). The half-life is the time after which, during incubation at a given temperature, the activity has fallen to 50% of the initial value (unit=min or h). The determination was carried out according to Example 9.

However, an increased thermal stability can also be derived for screening purposes from the so-called Ti value. The Ti value is calculated from the specific activity and a residual activity A15 according to the following formula:

Ti=A15/Ao

The activity A15 is the specific activity (based on the amount of FDH protein) which still exists after incubation of the enzymes for 15 minutes at an appropriate temperature (e.g. >50° C.). An increase in the Ti value of the mutant relative to the parents suggests an increased thermal stability.

Improved rec-enzymes are understood according to the claims as meaning particularly rec-enzymes which are more active and/or more selective (in respect of the reaction) and/or more stable under the reaction conditions used.

According to the invention, the claimed protein sequences and the nucleic acid sequences also include sequences which have a homology (exclusive of natural degeneracy) greater than 80%, preferably greater than 90%, 91%, 92%, 93% or 94%, particularly preferably greater than 95% or 96% and very particularly preferably greater than 97%, 98% or 99% to one of these sequences, provided the mode of action or the purpose of such a sequence is preserved. The expression ‘homology’ (or identity) as used here can be defined by the equation H (%)=[1−V/X]×100, where H denotes homology, X is the total number of nucleic acids/amino acids in the reference sequence and V is the number of different nucleic acids/amino acids in the sequence in question relative to the reference sequence. In any case, the expression ‘nucleic acids coding for amino acid sequences’ includes all sequences which appear possible according to the degeneracy of the genetic code.

The mean inactivating temperature T50 (also often denoted by Tm in the literature) is the temperature at which, after a given incubation period (in this case 20 min), the detectable activity has fallen to 50% of the initial value (unit =° C.).

The half-life is the time after which, during incubation at a given temperature, the activity has fallen to 50% of the initial value (unit =min or h).

The two parameters are related. One gives the (thermal) stability as a function of time and the other as a function of temperature.

If the inactivation of an enzyme is plotted in the form of the decrease in activity over time at different temperatures, the mean inactivating temperature can be determined from the curves obtained.

The literature references cited in this specification are incorporated herein by reference.

### EXAMPLES

Having generally described this invention, a further understanding can be obtained by reference to certain specific examples which are provided herein for purposes of illustration only and are not intended to be limiting unless otherwise specified.

### Example 1

### Genetic Engineering Methods

Unless indicated otherwise, all the genetic engineering methods used here are described by Sambrook et al. (1989) and are known to those skilled in the art. All the enzymes and corresponding buffers were used according to the manufacturers' instructions. The automatic sequencings with an ABI Sequencer (Applied Biosystems) were performed by SequiServe (Vaterstetten).

### Example 2

### Production of the Random Mutant Library

The error prone PCR was used to prepare the mutant libraries. The template used in the first generation was e.g. plasmid pBTac-FDH-C23S and those used in the subsequent generations were the plasmids with the selected more stable or more active mutants. The plasmids were isolated from E. coli using a QIAprep® Spin Miniprep Kit according to the manufacturer's instructions (Qiagen). pBTac2-specific primers were used as external primers in the error prone PCR preparations. The composition of the PCR preparation was as follows:

TABLE 5
Composition of the error prone PCR preparation

| Amount | Composition |
|---|---|
| 10 μl | 10× mutagenesis buffer (70 mM MgCl₂, 500 mM KCl, 0.1% |
| | (w/v) gelatin, 100 mM Tris-HCl pH 8.3 at 25° C.) |
| 10 μl | 10× mutagenesis dNTP mix (2 mM dGTP and dATP, 10 mM |
| | dTTP and dCTP) |
| 3-10 μl | 10× MnCl₂ (5 mM MnCl₂) |
| 2 fmol | template DNA (approx. 7.5 ng of 5.7 kb plasmid), |
| | e.g. pBTac-FDH-C23S |
| 40 pmol | upstream primer pBTacF1 |
| 40 pmol | downstream primer pBTacR1 or pBTacR2 |
| 1 μl | Taq polymerase (5 U μl⁻¹), Gibco |
| ad 100 μl | Millipore water |

The PCR programme used is listed in Table 6.

TABLE 6
Error prone PCR programme

| Step 1 | 95° C. | 5 min |
|---|---|---|
| 2 | 94° C. | 1 min |
| 3 | 50° C. | 1 min |
| 4 | 72° C. | X min |
| 5 | 72° C. | 5 min |

Steps 2-4 were run 25-27 times.

The annealing temperature TA was determined via the DNA melting temperature (Tm) of the oligonucleotides. The time X for the DNA polymerase chain reaction was governed by the rule 1 kb=1 min. The preparations were covered with a layer of approx. 50 μl of mineral oil.

The PCR products were purified using the QIAquick® PCR Purification Kit (Qiagen) or a preparative agarose gel.

To achieve a high mutation rate in the first generation of the mutagenesis for increasing the specific activity, 1 μl of PCR product was withdrawn after the first PCR and used in a second analogous PCR instead of the FDH-C23S template.

### Example 3

### Oligonucleotides

TABLE 7
List of the oligonucleotides used

| Name: | Use: | Sequence: |
|---|---|---|
| PBTACF1 | error prone PCR | 5′ TGC CTG GCA GTT CCC TAC TC 3′ |
| PBTACF2 | error prone PCR | 5′ CGT TTC ACT TCT GAG TTC GG 3′ |
| | sequencing | |
| PBTACR1 | error prone PCR | 5′ GGT ATG GCT GTG CAG GTC GT 3′ |
| PBTACR2 | error prone PCR | 5′ CGA CAT CAT AAC GGT TCT GG 3′ |
| | sequencing | |
| PBTACR3 | error prone PCR | 5′ TCA TCG GCT CGT ATA ATG TG 3′ |
| | sequencing | |
| F285S-F1 | site-directed | 5′- GGT GAT GTT TGG TCC CCA CAA CCA GCT CCA AAG -3′ |
| | mutagenesis | |
| F285S-R1 | site-directed | 5′- GGA GCT GGT TGT GGG GAC CAA ACA TCA CCG TA -3′ |
| | mutagenesis | |

### Example 4

### Site-Specific Mutagenesis

The specific introduction of point mutations was effected using the overlapping PCR method of Ho et al. (1989) or the QuickChange® Site-directed Mutagenesis Kit from Stratagene according to the manufacturer's instructions.

### Example 5

### In vitro Recombination by the Staggered Extension Process (StEP)

An in vitro recombination was carried out to combine the best mutants from the evolution of FDH-C23S and FDH-C23S/C262A. The SM-1eA6, SM-2 pC7, DM-3bE10 and DM-2hG12 mutants were recombined by means of StEP (H. Zhao, L. Giver, Z. Shao, J. Affholter, F. Arnold (1998) Molecular evolution by staggered extension process (StEP) in vitro recombination, Nat. Biotechnol. 16, 258-261).

TABLE 8
Composition of the recombination preparation

| Amount | Composition |
|---|---|
| 5 μl | 10× buffer (500 mM KCl, 200 mM Tris-HCl pH 8.4) |
| 5 μl | 10× dNTP mix (dGTP, dATP, dTTP and dCTP, all |
| | 2 mM) |
| 1.5 μl | 50 mM MgCl₂ |
| 0.075 pmol | template DNA, each mutant present in the same |
| | proportions |
| 7.5 pmol | upstream primer PBTACF1 |
| 7.5 pmol | downstream primer PBTACR1 |
| 0.5 μl | Taq polymerase (5 U μl⁻¹), Gibco |
| ad 50 μl | Millipore water |

TABLE 9
StEP programme

| Step 1 | 95° C. | 10 min |
|---|---|---|
| 2 | 94° C. | 30 sec |
| 3 | 50° C. | 10 sec |

Steps 2-3 were run 80 times.

The recombined FDH-DNA fragments were first incubated with the restriction enzyme DpnI in order to remove the parent DNA fragments from the preparations and thus minimize the proportion of parent clones in the mutant library. To facilitate cloning of the recombined fragments into vector pBTac, a restriction with EcoRI and PstI was then carried out overnight at 37° C. The EcoRI- and PstI-restricted FDH fragments were separated in an agarose gel and the FDH bands were isolated using the QlAquick® Gel Extraction Kit (Qiagen) according to the manufacturer's instructions and, as previously, cloned into pBTac2 and transformed in E. coli JM101. The resulting mutant library was screened for increased stability by the screening method described in Example 6.

### Example 6

### Method of Screening for Increased Stability

To screen for increased stability, the cells were cultured and the cell-free crude extracts prepared according to Example 7.2. The FDH muteins isolated in this way were first checked for stability (by the method of L. Giver, A. Gershenson, P.-O. Freskgard, F. Arnold (1998) Directed evolution of a thermostable esterase, Proc. Natl. Acad. Sci. USA 95, 12809-12813). This was done by first determining the initial activities Ao. 75 μl of FDH assay (see Example 7.2 for composition) were added to 75 μl of cell-free crude extract in a microtitre plate and the formation of NADH was monitored at 340 nm and 30° C. in a microtitre plate reader (Spectramax® Plus, MWG Biotech). 100 μl aliquots of crude extract were then transferred to PCR sample vessels in the 96 format and incubated for 15 min in a thermoblock of a PCR apparatus (Primus 96®, MWG Biotech) at a given temperature. The samples were then cooled on ice for 15 min and, if necessary, centrifuged (10 min, 4000 rpm) to separate off precipitated protein. 75 μl of the supernatant were used to determine the residual activity A15. The quotient of residual activity A15 to initial activity Ao is a measure of the stability of the muteins.

### Example 7

### Selection of the Clones with Increased Activity

7.1 Qualitative Selection

Culture of the Cells

The transformed cells were streaked on LB^{amp} agar plates and incubated overnight at 37° C. to give colonies with a diameter of approx. 1 mm.

Embedding of the Cells

The colonies were then covered with a layer of agar (1.6% agar, 100 mM KPᵢ pH 7.5, 0.2% Triton X-100, 10 mM EDTA). Before this, the agar solution had to be cooled to a temperature not exceeding 65° C. After the agar had solidified, it was washed five times with digesting solution (100 mM KPᵢ pH 7.5, 0.2% Triton X-100, 10 mM EDTA) and five times with washing solution (100 mM KPᵢ pH 7.5) (height of liquid in the plates: 5 mm).

Activity Staining

For staining, the plates were covered with a 2 mm deep layer of dye solution 1 (1.25 M formate; 0.2 gl⁻¹phenazine ethosulfate; 2 gl⁻¹ nitrotetrazolium blue chloride; 100 mM KPᵢ pH 7.5) and shaken in the dark for 10 min. Dye solution 2 (50 mM NAD) was then added in a proportion of 1 ml of dye solution 2 per 100 ml of dye solution 1 and shaken in the dark for approx. 15 min until the halos were clearly recognizable.

The solution was then poured off and the plates were briefly washed twice with water and left in the air to dry. When the plates were dry, the clones could be transferred with sterile toothpicks to 96-well plates filled with 200 μl of LB^{amp} medium. The cells were cultured overnight at 37° C. on a shaker. These plates served as master plates for the following quantitative selection.

7.2 Quantitative Selection

Culture of the Cells

Sterile deep-well plates in the 96 format were used for the culture. They were filled with 1.2 ml of LB^{amp} medium and inoculated with 50 μl of cell suspension from the master plates. After a growth phase of 4 h at 37° C., the cells were induced with 50 μl of an IPTG solution (20 mM IPTG in distilled water, sterile-filtered) and shaken at 140 rpm overnight at 30° C.

Cell Digestion, Preparation of the Cell-Free Crude Extracts

The 96-deep-well plates were then centrifuged (1600 g, 15 min, 20° C.), the supernatant was poured off, the cells were resuspended in 500 μl of buffer solution (10 mM KPᵢ pH 7.5), 1/10 vol. of digesting solution (2% Triton X-100, 10 mM KP, pH 7.5, 100 mM EDTA) was added and the plates were shaken for 1 h at 37° C. They were then centrifuged (1600 g, 15 min, 20° C.). 200 μl of the supernatant were then withdrawn and applied to the affinity chromatography material.

Purification by Means of Affinity Chromatography

Red sepharose (Procion Red HE-3B, DyStar, Frankfurt; bound to Streamline AC, Pharmacia) (U. Reichert, E. Knieps, H. Slusarczyk, M.-R. Kula, J. Thömmes (2001) Isolation of a recombinant formate dehydrogenase by pseudo affinity expanded bed adsorption, J. Biochem. Biophys. Methods, in press) was regenerated prior to use. This was done by washing it with regenerating solution 1 (1 M NaCl, 25% ethanol) and then with regenerating solution 2 (4 M urea, 0.5 M NaOH). It was then equilibrated with 10 mM KPᵢ pH 7.5. 200 μl of the supernatant of the cell digesting solution were transferred to a 96-well PCR plate (Roth) containing 10 μl of regenerated red sepharose. The PCR plate was then sealed with PCR strips and shaken slowly on an overhead shaker for 1 h at 20° C. The plate was then briefly centrifuged (1600 g, 1 min, 20° C.) and the supernatant was sucked off. The red sepharose was then washed ten times with washing solution 1 (40 mM NaCl, 40 mM NaHSO₄, 100 mM KPᵢ pH 7.5) and twice with washing solution 2 (100 mM KPᵢ pH 7.5). Elution was then carried out with the eluting solution (15 mM NAD, 100 mM KP? pH 7.5). This was done by adding 200 μl of the eluting solution, sealing the PCR plate and shaking it slowly on an overhead shaker for 1 h at 20° C. The plate was then briefly centrifuged again (1600 g, 1 min, 20° C.) and the supernatant was used to determine the volume activity and the protein concentration.

Determination of the Specific Activity

The volume activity was determined in microtitre plates using the Thermomax plus microtitre plate photometer (Molecular Devices). This was done by adding 25 μl of the eluted formate dehydrogenase to 75 μl of buffer (100 mM KPᵢ pH 7.5). Shortly before the measurement, 100 μl of activity assay (0.5 M Na formate, 4 mM NAD, 100 mM KPᵢ pH 7.5) are added to each well and the volume activity is determined.

The protein concentration was likewise determined in microtitre plates. Calibration was effected using a calibration curve with BSA (fraction V). 150 μl of 1.2×Bradford's solution were added to 50 μl of the eluted formate dehydrogenase and the protein concentration was determined.

The specific activity Vₘₐₓ[U mg⁻¹] is calculated by dividing the volume activity by the protein concentration. The turnover number k_{cat} [s⁻¹] can be calculated using the known molecular weight of formate dehydrogenase (40,370 g mol⁻¹):

k_{cat}=6×10⁻⁴Vₘₐₓ×Mw

### Example 8

### Purification of the FDH Muteins from E. coli JM101

The purification of the heterologously overexpressed FDH muteins was performed after cell digestion in one step:

1. Cell digestion: 40% cell suspension with 50 mM KPᵢ (pH 7.5) was digested by means of ultrasound.

2. Affinity chromatography by the batch method with red sepharose analogously to the method described above.

### Example 9

### Determination of the Half-Life

To determine the half-life, the enzyme solutions in 100 mM KPᵢ pH 7.5 were brought to the same volume activities and volumes in order to assure equal surface/volume ratios. The volume activity indicates the photometrically determined enzyme activity per volume of enzyme solution (units/ml). One unit is defined as the amount of enzyme which allows the reduction of 1 μmol of NAD, measured at the change of extinction at 340 nm, per minute at 30° C. and pH 7.5. The samples were incubated, e.g. at a given temperature ranging from 46° C. to 62° C., and aliquots were withdrawn at different times for determination of the volume activity.

The slope k can be determined by linear regression from a logarithmic plot of the volume activity and can be used to calculate the half-life t_{1/2}:

t_{1/2}=ln2/k

Obviously, numerous modifications and variations of the present invention are possible in light of the above teachings. It is therefore to be understood that within the scope of the appended claims, the invention may be practiced otherwise than as specifically described herein.

This application is based on German Patent Application Serial No. 101 46 589.0, filed on Sep. 21, 2001, and incorporated herein by reference.

1. A mutant of the wild-type rec-FDH from Candida boidinii which is more stable and/or more active as compared to said wild-type rec-FDH from Candida boidinii, wherein the mutant contains (a) the amino acid exchange C23S or C23S/C262A and (b) one or more amino acid exchanges selected from the group consisting of E18D, K35R, D149E, E151D, R178S, R178G, K206R, F285Y, F285S, T315N and K356E.

2. The mutant of claim 1, which contains the C23S amino acid exchange.

3. A mutant of the native wild-type FDH from Candida boidinii which is more stable and/or more active as compared to said wild-type FDH from Candida boidinii, wherein the mutant contains (a) the amino acid exchange C23S or C23S/C262A and (b) one or more amino acid exchanges selected from the group consisting of E18D, K35R, D149E, E151D, R178S, R178G, K206R, F285Y, F285S, T315N and K356E.

4. The mutant of claim 1, which contains the C23S amino acid exchange.

5. An amino acid sequence having FDH activity which is more stable and/or more active as compared to the wild-type rec-FDH and the native wild-type enzyme from Candida boidinii and which contain one or more of the following amino acid exchanges: 18D, 35R, 149E, 151D, 178S, 178G, 206R, 285Y, 285S, 315N and 356E, the exchanges taking place in the corresponding equivalent positions in the sequence.

6. A nucleic acid encoding the mutant of claim 1.

7. A nucleic acid encoding the mutant of claim 3.

8. A nucleic acid encoding the amino acid sequence of claim 5.

9. A plasmid, vector or a microorganism containing one or more nucleic acids according to claim 6.

10. A plasmid, vector or a microorganism containing one or more nucleic acids according to claim 7.

11. A plasmid, vector or a microorganism containing one or more nucleic acids according to claim 8.

12. A primer selected from the group consisting of SEQ ID NO: 25, 26, 27, 28, 29, and 30.

13. A process for the preparation of improved rec-FDHs from a nucleic acidscoding for a rec-FDH according to claim 1, comprising:
a) mutagenizing said nucleic acid,
b) cloning the mutagenized nucleic acid into a suitable vector and transferring the vector into a suitable expression system, and
c) detecting and isolating an improved rec-FDH.

14. rec-FDHs or nucleic acids coding therefor, obtainable according to the process of claim 13.

15. A method of preparing chiral compounds, comprising reacting a starting material in the presence of the mutant according to claim 1 and producing a chiral compound.

16. The method of claim 15, wherein the chiral compound is an alcohol or an amino acid.

17. A method of preparing chiral compounds, comprising reacting a starting material in the presence of the mutant according to claim 3 and producing a chiral compound.

18. The method of claim 17, wherein the chiral compound is an alcohol or an amino acid.

19. A method of preparing chiral compounds, comprising reacting a starting material in the presence of the amino acid sequences according to claim 5 and producing a chiral compound.

20. The method of claim 19, wherein the chiral compound is an alcohol or an amino acid.

21. A method of preparing NADH, comprising reacting formate with the mutant of claim 1 in the presence of NAD⁺.

22. A method of preparing NADH, comprising reacting formate with the mutant of claim 3 in the presence of NAD⁺.

23. A method of preparing NADH, comprising reacting formate with the amino acid sequence of claim 5 in the presence of NAD⁺.

24. A cell transformed with the nucleic acid of claim 6.

25. A cell transformed with the nucleic acid of claim 7.

26. A cell transformed with the nucleic acid of claim 8.

27. A cell containing a cloned gene for a dehydrogenase and a cloned gene for a rec-FDH.

28. The cell according to claim 27, wherein the rec-FDH is from Candida boidinii.

29. A cell containing a cloned gene which encodes the mutant of claim 1.

30. A cell containing a cloned gene which encodes the mutant of claim 3.

31. A cell containing a cloned gene which encodes the amino acid sequence of claim 5.

32. A method of identifying more active mutants of an NAD- or NADP-dependent dehydrogenase, comprising a quantitative screening method for determination of the activity, said method consisting of the following steps:
a) contacting equal aliquots of a cell digesting solution of the mutants to be compared with equal amounts of an affinity chromatography material,
b) separating the affinity chromatography material from the non-adhering constituents,
c) eluting the muteins adhering to the affinity chromatography material, and
d) determining the volume activity and protein concentration, and hence the specific activity.

33. The method according to claim 32, wherein the enzyme is an FDH.


SEQUENCE LISTING (43 sequences)

SEQ ID NO: 1 - DNA, 1095 bp, ARTIFICIAL SEQUENCE
  SYNTHETIC DNA
atgaagattgtcttagttctttatgatgctggtaagcacgctgctgatgaagaaaaattatatggttctactgaaaataaattaggtattgctaattggttaaaagatcaaggtcatgaactaattactacttctgataaagaaggtgaaacaagtgaattggataaacatatcccagatgctgatattatcatcaccactcctttccatcctgcttatatcactaaggaaagacttgacaaggctaagaacttaaaattagtcgttgtcgctggtgttggttctgatcacattgatttagattatattaatcaaacaggtaagaaaatctcagtcctggaagttacaggttctaatgttgtctctgttgctgaacacgttgtcatgaccatgcttgtcttggttagaaatttcgttccagcacatgaacaaattattaaccacgattgggaggttgctgctatcgctaaggatgcttacgatatcgaaggtaaaactatcgctaccattggtgctggtagaattggttacagagtcttggaaagattactcccatttaatccaaaagaattattatactacgattatcaagctttaccaaaagaagctgaagaaaaagttggtgctagaagagttgaaaatattgaagaattagttgctcaagctgatatcgttacagttaatgctccattacacgcaggtacaaaaggtttaattaataaggaattattatctaaatttaaaaaaggtgcttggttagtcaataccgcaagaggtgctatttgtgttgctgaagatgttgcagcagctttagaatctggtcaattaagaggttacggtggtgatgtttggttcccacaaccagctccaaaggatcacccatggagagatatgagaaataaatatggtgctggtaatgccatgactcctcactactctggtactactttagacgctcaaacaagatacgctgaaggtactaaaaatattttggaatcattctttaccggtaaatttgattacagaccacaagatattatcttattaaatggtgaatacgttactaaagcttacggtaaacacgataagaaataa

SEQ ID NO: 2 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 3 - DNA, 1095 bp, ARTIFICIAL SEQUENCE; identical to SEQ ID NO: 1
  SYNTHETIC DNA

SEQ ID NO: 4 - protein, 364 aa, ARTIFICIAL SEQUENCE; identical to SEQ ID NO: 2
  SYNTHETIC DNA

SEQ ID NO: 5 - DNA, 1095 bp, ARTIFICIAL SEQUENCE; identical to SEQ ID NO: 1
  SYNTHETIC DNA

SEQ ID NO: 6 - protein, 364 aa, ARTIFICIAL SEQUENCE; identical to SEQ ID NO: 2
  SYNTHETIC DNA

SEQ ID NO: 7 - DNA, 1095 bp, ARTIFICIAL SEQUENCE; identical to SEQ ID NO: 1
  SYNTHETIC DNA

SEQ ID NO: 8 - protein, 364 aa, ARTIFICIAL SEQUENCE; identical to SEQ ID NO: 2
  SYNTHETIC DNA

SEQ ID NO: 9 - DNA, 1095 bp, ARTIFICIAL SEQUENCE; identical to SEQ ID NO: 1
  SYNTHETIC DNA

SEQ ID NO: 10 - protein, 364 aa, ARTIFICIAL SEQUENCE; identical to SEQ ID NO: 2
  SYNTHETIC DNA

SEQ ID NO: 11 - DNA, 1095 bp, ARTIFICIAL SEQUENCE; identical to SEQ ID NO: 1
  SYNTHETIC DNA

SEQ ID NO: 12 - protein, 364 aa, ARTIFICIAL SEQUENCE; identical to SEQ ID NO: 2
  SYNTHETIC DNA

SEQ ID NO: 13 - DNA, 1095 bp, ARTIFICIAL SEQUENCE; identical to SEQ ID NO: 1
  SYNTHETIC DNA

SEQ ID NO: 14 - protein, 364 aa, ARTIFICIAL SEQUENCE; identical to SEQ ID NO: 2
  SYNTHETIC DNA

SEQ ID NO: 15 - DNA, 1095 bp, ARTIFICIAL SEQUENCE; identical to SEQ ID NO: 1
  SYNTHETIC DNA

SEQ ID NO: 16 - protein, 364 aa, ARTIFICIAL SEQUENCE; identical to SEQ ID NO: 2
  SYNTHETIC DNA

SEQ ID NO: 17 - DNA, 1095 bp, ARTIFICIAL SEQUENCE; identical to SEQ ID NO: 1
  SYNTHETIC DNA

SEQ ID NO: 18 - protein, 364 aa, ARTIFICIAL SEQUENCE; identical to SEQ ID NO: 2
  SYNTHETIC DNA

SEQ ID NO: 19 - DNA, 1095 bp, ARTIFICIAL SEQUENCE; identical to SEQ ID NO: 1
  SYNTHETIC DNA

SEQ ID NO: 20 - protein, 364 aa, ARTIFICIAL SEQUENCE; identical to SEQ ID NO: 2
  SYNTHETIC DNA

SEQ ID NO: 21 - DNA, 1095 bp, ARTIFICIAL SEQUENCE; identical to SEQ ID NO: 1
  SYNTHETIC DNA

SEQ ID NO: 22 - protein, 364 aa, ARTIFICIAL SEQUENCE; identical to SEQ ID NO: 2
  SYNTHETIC DNA

SEQ ID NO: 23 - DNA, 1095 bp, ARTIFICIAL SEQUENCE; identical to SEQ ID NO: 1
  SYNTHETIC DNA

SEQ ID NO: 24 - protein, 364 aa, ARTIFICIAL SEQUENCE; identical to SEQ ID NO: 2
  SYNTHETIC DNA

SEQ ID NO: 25 - DNA, 20 bp, ARTIFICIAL SEQUENCE
  SYNTHETIC DNA
tgcctggcagttccctactc

SEQ ID NO: 26 - DNA, 20 bp, ARTIFICIAL SEQUENCE
  SYNTHETIC DNA
cgtttcacttctgagttcgg

SEQ ID NO: 27 - DNA, 20 bp, ARTIFICIAL SEQUENCE
  SYNTHETIC DNA
ggtatggctgtgcaggtcgt

SEQ ID NO: 28 - DNA, 20 bp, ARTIFICIAL SEQUENCE
  SYNTHETIC DNA
cgacatcataacggttctgg

SEQ ID NO: 29 - DNA, 20 bp, ARTIFICIAL SEQUENCE
  SYNTHETIC DNA
tcatcggctcgtataatgtg

SEQ ID NO: 30 - DNA, 33 bp, ARTIFICIAL SEQUENCE
  SYNTHETIC DNA
ggtgatgtttggtccccacaaccagctccaaag

SEQ ID NO: 31 - DNA, 32 bp, ARTIFICIAL SEQUENCE
  SYNTHETIC DNA
ggagctggttgtggggaccaaacatcaccgta

SEQ ID NO: 32 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEERVGARRVENIEELVAQADIVTVNAPLHAGTKGLINKELLSKFKKGAWLVNTARGAIAVAEDVAAALESGQLRGYGGDVWFPQPAPKDHPWRDMRNKYGAGNAMTPHYSGNTLDAQTRYAEGTKNILESFFTGKFDYRPQDIILLNGEYVTEAYGKHDKK

SEQ ID NO: 33 - protein, 364 aa, ARTIFICIAL SEQUENCE
  SYNTHETIC DNA
MKIVLVLYDAGKHAADEDKLYGSTENKLGIANWLRDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAIAVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 34 - protein, 364 aa, ARTIFICIAL SEQUENCE
  SYNTHETIC DNA
MKIVLVLYDAGKHAADEDKLYGSTENKLGIANWLRDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWDVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAIAVAEDVAAALESGQLRGYGGDVWYPQPAPKDHPWRDMRNKYGAGNAMTPHYSGTTLDAQTRYAEGTKNILESFFTGKFDYRPQDIILLNGEYVTKAYGKHDKK

SEQ ID NO: 35 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 36 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYGVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 37 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHEWEVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 38 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWDVAAIAKDAYDIEGKTIATIGAGRIGYSVLERLLPFNPKELLYYDYQALPKEAEERVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGNTLDAQTRYAEGTKNILESFFTGKFDYRPQDIILLNGEYVTKAYGKHDKK

SEQ ID NO: 39 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWDVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 40 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWDVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTEAYGKHDKK

SEQ ID NO: 41 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWDVAAIAKDAYDIEGKTIATIGAGRIGYSVLERLLPFNPKELLYYDYQALPKEAEERVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTEAYGKHDKK

SEQ ID NO: 42 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYVTKAYGKHDKK

SEQ ID NO: 43 - protein, 364 aa, ARTIFICIAL SEQUENCE
  SYNTHETIC DNA
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWDVAAIAKDAYDIEGKTIATIGAGRIGYSVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWSPQPAPKDHPWRDMRNKYGAGNAMTPHYSGTTLDAQTRYAEGTKNILESFFTGKFDYRPQDIILLNGEYVTKAYGKHDKK